(19) United States
(12) Patent Application Publication
(10) Pub. No.: US 2007/0021751 A1
(43) Pub. Date: Jan. 25, 2007

(54) Bone anchor

(75) Inventors: Clive Reay-Young, Harrogate (GB); Seth Hootstein, New York, NY (US)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/131,603

(22) Filed: May 17, 2005

Related U.S. Application Data
(60) Provisional application No. 60/571,981, filed on May 17, 2004.

(51) Int. Cl. A61B 17/58

(52) U.S. Cl. 606/72

(57) ABSTRACT

A bone anchor adapted to secure soft tissue to bone. The anchor includes a first body section and a second body section proximal to the first body section. The first section has a non-circular cross section such that once the implant is inserted into a shaped bone tunnel it may be secured in the tunnel by rotation from its initial insertion position to a second position. The dissimilarity of the non-circular cross section of the proximal end of the first section prevents removal of the bone anchor from the bone tunnel. Additionally, a suture may be threaded through a hole in the second section. Once the anchor is secured in place with an attached suture, the free ends of the suture may be threaded through soft tissue and tied down to secure the soft tissue to bone.

### BACKGROUND OF THE INVENTION

1. Technical Field

This invention relates to an apparatus and method for attaching soft tissue to bone. More specifically, the invention relates to implants used in conjunction with a suture for attachment of soft tissue to bone.

2. Description of the Prior Art

Surgical procedures for securing soft tissue to bone are becoming more prevalent. For example, it is known in the prior art to repair the rotator cuff by reattaching the rotator cuff tissue to the humerus head. This may be carried out by puncturing the torn cuff and drilling prethreaded soft tissue fasteners, e.g., anchor screws, into the head of the humerus bone. The sutures are then passed through the tissues. After the suture strands are passed through the tissue, they are knotted and tied together to secure the rotator cuff to the humerus head.

Other implants and techniques for securing soft tissue are described in U.S. Pat. Nos.: 6,117,161; 6,146,407; 6,491,714; 6,551,343; 6,547,800; 6,610,080. However, a drawback to some of the prior art techniques is that the implanted anchors may loosen or migrate over time. As the anchor loosens, the joint destabilizes. Another drawback to some of the known techniques is that they are overly complicated or involve too many steps. An improved bone anchor is desired. In particular, there is a need for a bone anchor that has less potential to loosen or migrate than the prior art devices.

### SUMMARY OF THE INVENTION

This invention comprises an apparatus and method for securing soft tissue to bone.

In a first aspect of the invention, a bone anchor comprises a first section and a second section proximal to the first section. The first section has an elongate body, a proximal end and a distal end, with the proximal end of the first section having a non-circular cross section. The proximal end of the first section is adapted to be placed in an opening formed in cortical bone. The second section in communication with the first section is provided with an aperture and an external channel extending from said aperture. Following placement of the bone anchor in the cortical bone opening, the bone anchor is rotated from a first position to a second position. Rotation of the body prevents removal of the first section from the cortical bone opening. Additionally, the body of the first section may be smooth, continuous, without projections, non expandable, and the like. It is also not necessary for the first section to have sharp edges, ribs, or arm members.

In another aspect of the invention, a method of securing a tissue to a bone body is provided. The method comprises forming an elongate passageway having a proximal cortical region and a distal cancellous bone region. The cortical bone region has an opening. The method further comprises inserting a bone anchor into the passageway. The bone anchor includes a first section and a second section proximal to the first section wherein the first section of the bone anchor has a cross sectional shape that, when aligned with the opening, is insertable into the passageway. The bone anchor is inserted into the passageway such that the first section of the bone anchor is positioned distal to the cortical bone region and within the cancellous bone region. The method further comprises rotating the bone anchor from a first position to a second position, such that the bone anchor may not be withdrawn from the passageway due to the first section of the anchor being unaligned with the opening in the cortical bone region. The method additionally comprises affixing tissue to the bone anchor. The bone anchor may be configured as described herein. In another aspect of the invention, the above affixing step may include tying the tissue to the bone anchor using a suture.

In yet another aspect of the present invention, a kit is provided. The kit includes a bone anchor, a suture, and a driver instrument to manipulate the bone anchor.

Other features and advantages of this invention will become apparent from the following detailed description of the presently preferred embodiment of the invention, taken in conjunction with the accompanying drawings.

### BRIEF DESCRIPTION OF THE DRAWINGS

FIG. 1 is a perspective view of a bone anchor according to the preferred embodiment of this invention, and is suggested for printing on the first page of the issued patent.

FIG. 2 is a top view of the bone anchor of FIG. 1.

FIG. 3 is a side view of the bone anchor of FIG. 1.

FIG. 4 is a front view of the bone anchor of FIG. 1.

FIGS. 5A-5C are illustrations of a bone anchor being inserted and locked in a bone body.

FIG. 6 illustrates a driver instrument and a bone anchor held at the distal end of the driver instrument.

### DESCRIPTION OF THE PREFERRED EMBODIMENT

Before the present invention is described in detail, it is to be understood that this invention is not limited to particular embodiments and applications described and as such may, of course, vary. Also, reference to a singular item, includes the possibility that there are plural of the same items present. More specifically, as used herein and in the appended claims, the singular forms “a,” “and,” “said”” and “the” include plural referents unless the context clearly dictates otherwise.

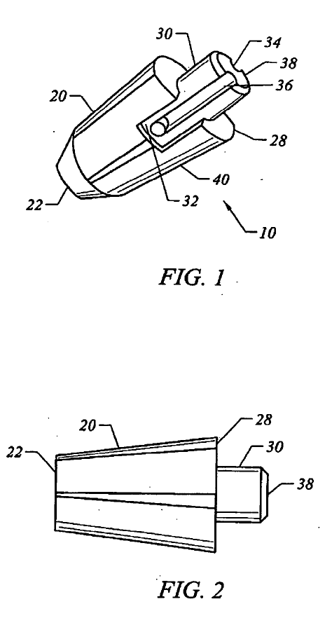

Referring to FIG. 1, a fixation device for attaching or securing soft tissue to bone is in the form of a bone anchor 10, which includes a first section 20 and a second section 30. The first section 20 has a distal end 22 with a circular or near circular cross section, and a proximal end 28 with a non-circular or oblong cross section. The cross section of the proximal end 28 is larger than the cross section of the distal end 22. An exterior surface of the first section has a conical shape extending from the proximal end 28 to the distal end 22. The exterior surface is relatively smooth, and does not include any significant impedances, ribs, arms, sharp edges, or protrusions.

The second section 30 has a distal end 32 with a circular or near circular cross section, and a proximal end 38 with a circular or near-circular cross section. The cross section of the distal end 32 is approximately equal to the cross section of the proximal end 38. Adjacent to the distal end 32, the second section 30 has an opening 40 extending there through. In addition, an exterior surface of the second section 30 has a pair of diametrically opposed channels 34, 36. The opening 40 extends through the second section from the first channel 34 to the second channel 36.

As shown in FIGS. 1 and 4, the distal end 22 of the first section 20 has a round or near round cross sectional shape with a defined cross section. If the distal end 22 is rotated along its axis, the cross sectional shape would remain uniform. However, the proximal end 28 of the first section 20 has a larger cross sectional area than the distal end 22 of the first section. In addition, the cross sectional area of the proximal end 28 is non-circular. Both the distal end 22 and the proximal end 28 of the first section are adapted to fit within an opening in the cortical bone that has a shape similar to that of the proximal end 28. Rotation of the second section by an operator from a first position to a second position will rotate both the second section 30 and the first section 20. Additionally, since the cross-sectional area of the proximal end 28 of the first section 20 is non-circular, rotation of the first section 20 will prevent the proximal end 28 of the first section 20 from being removed from the opening. Accordingly, once the proximal end of the first section is placed through an opening in the cortical bone and is rotated therein, the rotated cross sectional area of the proximal end of the first section will prevent removal of the proximal end from the opening, thereby securing the bone anchor in place.

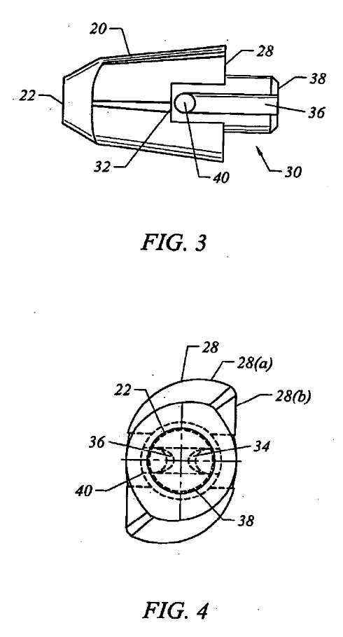

Referring to FIG. 3, opening 40 in the second section 30 is visible. This opening 40 is adapted to receive a securing element (e.g., a suture). Preferably, prior to insertion of the anchor into the bone tunnel, a securing element is threaded through the opening 40 in the second section 30. Legs of the threaded securing elements are placed in the diametrically opposed channels 34, 36 so that the securing element does not interfere with insertion of the anchor into the tunnel. This section may include additional opening and channels to accommodate additional sutures.

Preferably, after the bone anchor is threaded with a suture, a tunnel is formed in a section of bone. The tunnel is preferably sized and shaped with respect to the cross-section area of the proximal end 28 of the first section 20. The bone tunnel or passageway entrance should match this cross-sectional area or shape. A passageway of this nature may be created using a drill and punch or perhaps, only a punch. Other techniques to create the entrance and passageway should be apparent to those skilled in the relevant art.

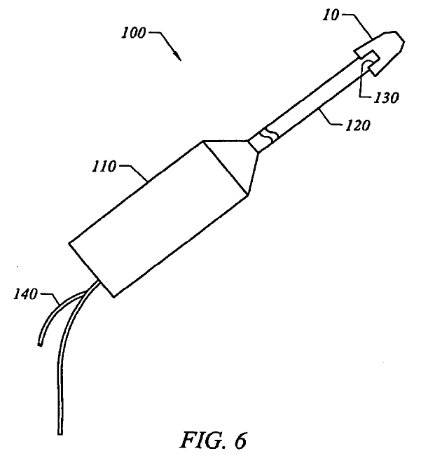

After the tunnel is created, the bone anchor is inserted into the tunnel. A driver instrument may be provided to facilitate insertion of the anchor into the bone. As shown in FIG. 6, a driver 100 includes a handle portion 110 and a shaft 120. The shaft has a distal end that is adapted to interlock with the bone anchor 10. Arms 130 are adapted to removably interlock with corresponding recesses in anchor 10. The driver instrument may also have an internal lumen to receive suture legs extending from the anchor. The suture legs 140 are shown exiting the proximal end of the handle in FIG. 6. Accordingly, a surgeon may conveniently manipulate the preloaded anchor into a target opening. Such an opening may be any opening in a bone body, preferably an opening in the hard cortical bone layer. The opening should be sized to match the profile of the anchor at its largest cross section. For example, in the above described device, the region of the anchor having the largest profile or cross section is the proximal end 28 of the first section. Such passageways may be made with, e.g., punches and drills.

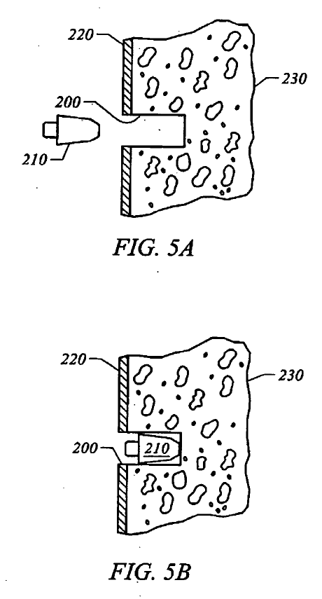

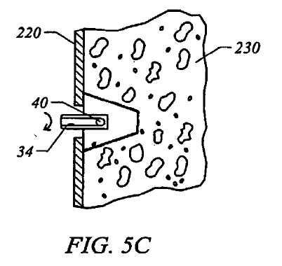

FIGS. 5A-5C illustrate insertion of the bone anchor in a tunnel. The first section or body 210 is manipulated beyond the cortical bone 220 and into the cancellous bone 230. Once in this first position as shown in FIG. 5B, the surgeon rotates the anchor into a second position as shown in FIG. 5C. The non circular (or oblong) anchor body displaces the cancellous bone 230 and other tissue as the anchor is rotated. The anchor is preferably rotated between 45 and 135 degrees and more preferably about 90 degrees from the first position. The non-circular cross-sectional area of the proximal end 28 of the first section prevents removal of the anchor 210 from the tunnel when the anchor is in the second position. Also, the flat surface 28B shown in FIG. 4 inhibits the anchor from returning or migrating towards the first position. In order to remove the anchor from the tunnel, the anchor would need to be rotated from the second position to the first position. Accordingly, the anchor is held or locked in position under the cortical bone. Additionally, the securing element or suture is held in position via a through-hole, eyelet, or another type of suture receiver 40.

The driver is then removed leaving the anchor in the passage and sutures extending there from. The free ends of the suture are then threaded through soft tissue such as ligaments, tendons, muscles, etc. knotted and tied down to the bone surface. In this manner, various soft tissues may be properly anchored. For example, the glenoid humerus and glenoid labrum ligament complexes may be secured to the proper bone body.

As shown in FIG. 5C, a portion of the proximal end of the second section may remain external to the tunnel. Additionally, a portion of the second section is located under the cortical bone layer and within the cancellous bone layer.

As indicated above, the bone anchor 10 is a non-expanding implant that locks with the cortical bone upon rotation. It typically has a conical shape with a first section having a non-circular cross section that enables locking upon rotation of the anchor. However, the bone anchor 10 may include a wide variety of alternative shapes. For example, the first section may have a cylindrical shape or the distal end of the first section may have a non-circular cross section that enables locking of the anchor to the cortical bone upon rotation from a first position to a second position. Although not preferred, the bone anchor in some embodiments may include additional features such as threads and protrusions to facilitate insertion and biting into the bone.

The bone anchor is preferably made from a material that is biocompatible or bioabsorbable. The bone anchor may be manufactured by, for example, injection molding, machining, or other manufacturing techniques known to those skilled in art. The bone anchor 10 may be made of an osteo-conductive material, or osteo-compatible material. Poly-L-lactic acid (PLLA)/tricalcium phosphate and PLLA/hydroxylapatite are examples of materials that may be used to form the bone anchor in accordance with the present invention.

The bone anchor enables insertion of a smooth continuous exterior surface through the cortical bone and into the cancellous bone. Protrusions, ribs, and elements along the exterior surface are not necessary to secure the anchor to the bone. It is the profile of the proximal end of the first section of the anchor and rotation thereof that enables the anchor to lock into a secure position. There is no requirement for an additional machined element, step, or mechanical component to lock the anchor to the cortical bone. There is no requirement for ribs or bladed arms. In addition, the second section of the anchor has an aperture adapted to receive a suture there through, and a pair of diametrically opposed longitudinal channels adapted to receive the suture and to hold the legs of the suture adjacent to the aperture.

It will be appreciated that, although specific embodiments of the invention have been described herein for purposes of illustration, various modifications may be made without departing from the spirit and scope of the invention. In particular, the bone anchor is shown as one unit with two sections, wherein the sections may be integrated as a solid unit, or they may be separably combinable. The anchor may be manufactured through injection molding or machined. Also, instead of a through-hole, the second section may include a loop, ring, eye, or indent member to receive a leg of a securing element.

In addition, the implant is shown having a conical shape. However, the invention should not be limited to the shape shown in the figures. The shape of the implant may come in many shapes wherein the exterior surface is relatively free of expandable elements or encumberances, and the profile of the proximal end is non-circular and enables locking of the implant upon rotation from an insertion profile. Accordingly, the scope of protection of this invention is limited only by the following claims and their equivalents.

All publications, patents and patent applications mentioned in this document are incorporated by reference in their entirety.

We claim:

1. A bone anchor comprising:
a first section having a proximal end and a distal end;
said proximal end of said first section having a non-circular cross section and adapted to be placed into an opening in cortical bone;
a second section in communication with and proximal to said first section, said second section having an aperture and an external channel extending proximally from said aperture; and
said first section is adapted to be rotated from a first position to a second position following placement in said cortical bone opening, wherein said rotation prevents removal of said first section from said opening in cortical bone.

2. The bone anchor of claim 1, wherein said shape of said first section of said bone anchor is smooth, continuous, and rib-less.

3. The bone anchor of claim 2 wherein said first section has an elongate body with an oblong cross section.

4. The bone anchor of claim 1, wherein said first section has a varying cross section and wherein said non-circular cross section is at one location along an axis of said bone anchor.

5. The bone anchor of claim 4, wherein said channel is adapted to extend from said aperture to said proximal end of said second section.

6. The bone anchor of claim 5, wherein said aperture is adapted to receive a securing element.

7. The bone anchor of claim 6, wherein said channel is adapted to receive a leg of said securing element.

8. The bone anchor of claim 6, wherein said aperture with said securing element is adapted to reside within said opening in said cortical bone following placement of said securing element in said aperture and rotation of said proximal end of said first section in said cortical bone opening.

9. The bone anchor of claim 4, wherein said sections are comprised of a material selected from a group consisting of: osteo-compatible, osteo-conductive, and combinations thereof.

10. The bone anchor of claim 4, wherein said first and second sections are a single cohesive unit.

11. The bone anchor of claim 1, wherein said first section is comprised of an external surface having a characteristic selected from a group consisting of: a non-expandable, smooth, free of an encumbrance, and combinations thereof.

12. A method of securing a tissue to a bone body, said bone body comprising a hard outer cortical bone layer and an inner cancellous bone layer, said method comprising:
a.) forming an elongate passageway having a proximal cortical region and a cancellous bone region, said cortical bone region having an opening;
b.) inserting a bone anchor into said passageway, said bone anchor having a first section and a second section proximal to said first section wherein the first section of said bone anchor has a cross sectional shape that, when aligned with said opening, is insertable into said passageway, said bone anchor being inserted into said passageway such that said first section of said bone anchor is positioned distal to said cortical bone region and in said cancellous bone region;
c.) rotating said bone anchor from a first position to a second position, such that said bone anchor may not be withdrawn from said passageway due to said first section of said anchor being unaligned with said opening of said cortical bone region; and
d.) affixing tissue to the bone anchor.

13. The method of claim 12, wherein said second section of said bone anchor includes a through-hole and a pair of diametrically opposing external channels extending proximally from said through hole.

14. The method of claim 12, wherein said first and second sections are part of a single cohesive unit.

15. The method of claim 12, wherein said affixing includes tying said tissue to said bone anchor using a suture.

16. A surgical kit comprising:
a bone anchor as described in claim 1; and
a suture.

17. The kit of claim 16 further comprising a driver instrument having a distal end configured to removably interlock with a proximal end of the second section of the bone anchor.

18. The kit of claim 17 wherein said bone anchor is threaded with said suture through said opening.

19. The kit of claim 16 wherein said second section of said bone anchor comprises a recess, and said distal end of said driver instrument comprises an arm member that is removably received by said recess of said bone anchor.

20. The bone anchor of claim 1 wherein said bone anchor comprises a polymer matrix comprising tri-calcium phosphate.

21. The bone anchor of claim 1 wherein said second section comprises a second aperture for receiving a second suture.
